(19) United States
(12) Patent Application Publication
(10) Pub. No.: US 2026/0315020 A1
(43) Pub. Date: Oct. 15, 2026

(54) DEEP LEARNING MODEL FOR DIAGNOSING PLAQUE EROSION USING CORONARY COMPUTED TOMOGRAPHY ANGIOGRAPHY

(71) Applicant: The General Hospital Corporation, Boston, MA (US)

(72) Inventors: Ik-Kyung Jang, Boston, MA (US); Sangjoon Park, Daejeon (KR); Jong Chul Ye, Daejeon (KR)

(21) Appl. No.: 19/481,096

(22) PCT Filed: May 3, 2024
(86) PCT No.: PCT/US2024/027699

§ 371 Date: Nov. 3, 2025

Related U.S. Application Data
(60) Provisional application No. 63/499,840, filed on May 3, 2023.

(51) Int. Cl. A61B 6/50; A61B 6/00; A61B 6/03; A61B 6/46; G16H 20/00; G16H 30/40; G16H 50/20

(52) U.S. Cl. CPC A61B 6/504; A61B 6/032; A61B 6/461; A61B 6/5217; G16H 20/00; G16H 30/40; G16H 50/20

(57) ABSTRACT

Systems and methods of deep-learning diagnosis of plaque erosion using coronary computed tomography angiography (CTA). One system may include one or more electronic processors configured to receive a plurality of CTA images for a patient having acute coronary syndromes (ACS) and apply a machine learning model to the plurality of CTA images to determine a plaque erosion prediction.

### CROSS-REFERENCE TO RELATED APPLICATIONS

This application claims priority to U.S. Provisional Application No. 63/499,840, filed May 3, 2023. The entire contents of which is incorporated herein by reference.

### BACKGROUND

This disclosure is directed toward plaque erosion, and, in particular, diagnosing plaque erosion using coronary computed tomography angiography. Acute coronary syndromes (ACS) are the most common cause of death worldwide. Previous reports have revealed that plaque rupture was the underlying mechanism in the majority of cases.

The discussion above is merely provided for general background information and is not intended to be used as an aid in determining the scope of the claimed subject matter.

### SUMMARY

Various aspects of the present disclosure relate to systems and methods for diagnosing plaque erosion using coronary computed tomography angiography (CTA), and, in particular, to implementing a deep learning (DL) model for diagnosing plaque erosion using coronary CTA. In some configurations, the technology disclosed herein may provide methods and systems of developing and implementing a DL model that enables non-invasive diagnosis of plaque erosion using CTA in acute coronary syndromes (ACS) patients, which may lead to a major shift in the management of patients with ACS. Accordingly, in some configurations, the technology disclosed herein provides methods and systems that enable an accurate CT diagnosis of plaque erosion, such that, e.g., cardiologists (or other medical professionals) may provide tailored therapy without invasive procedures.

One configuration provides a system for implementing deep learning to noninvasively diagnose plaque erosion using coronary computed tomography angiography (CTA). The system may include one or more electronic processors. The one or more electronic processors may be configured to receive a plurality of CTA images for a patient having acute coronary syndromes (ACS). The one or more electronic processors may be configured to apply a machine learning model to the plurality of CTA images, where the machine learning model may be a modality-specific deep learning model trained using machine learning to diagnose plaque erosion using coronary CTA. The one or more electronic processors may be configured to determine a plaque erosion prediction based on application of the machine learning model to the plurality of CTA images. The one or more electronic processors may be configured to transmit the plaque erosion prediction for display via a display device.

In some configurations, the one or more electronic processors may be configured to: determine, based on the plaque erosion prediction, a recommended course of treatment for the patient; and transmit the recommended course of treatment for display via the display device.

In some configurations, the recommended course of treatment may include an antithrombotic therapy without stenting when the plaque erosion prediction indicates a first probability of plaque erosion; and the recommended course of treatment may include a percutaneous coronary intervention when the plaque erosion prediction indicates a second probability of plaque erosion, where the second probability of plaque erosion is lower than the first probability of plaque erosion.

In some configurations, the plaque erosion prediction may include at least one of a probability of plaque erosion or a probability of plaque rupture.

In some configurations, the machine learning model may include a composite transformer attention component and a momentum distillation component.

In some configurations, the machine learning model may include a spatial transformer and a sequential transformer.

In some configurations, the one or more electronic processors may be configured to determine the plaque erosion prediction by: applying a spatial transformer of the machine learning model to each of a plurality of CTA slices included in each of the plurality of CTA images to generate a corresponding feature vector for each of the plurality of CTA slices; and applying a sequential transformer of the machine learning model to each corresponding feature vector of each of the plurality of CTA slices to output a transformed feature, where the machine learning model may be configured to determine the plaque erosion prediction based on the transformed feature. In some instances, the machine learning model may be a classification model.

In some configurations, the one or more electronic processors may be configured to: determine, with the machine learning model, a slice-level prediction based on the plurality of CTA images; and determine, with the machine learning model, a patient-level prediction based on the plurality of CTA images, where the plaque erosion prediction may be based on the slice-level prediction and the patient-level prediction.

In some configurations, the one or more electronic processors may be configured to: access training data including a plurality of training CTA images, where at least a portion of the plurality of training CTA images may be annotated; and train, with the training data, the machine learning model using momentum distillation-enhanced self-supervised pre-training. In some instances, the machine learning model may use momentum distillation-enhanced self-supervised pre-training from a student network to enhance the performance along with the supervised learning.

Yet another configuration may provide a method for implementing deep learning to noninvasively diagnose plaque erosion using coronary computed tomography angiography (CTA). The method may include receiving, with one or more electronic processors, a plurality of medical images. The method may include applying, with the one or more electronic processors, a machine learning model to the plurality of medical images, where the machine learning model may be a modality-specific deep learning model including a spatial transformer and a sequential transformer. The method may include determining, with the one or more electronic processors, a plaque erosion prediction based on application of the machine learning model to the plurality of medical images. The method may include transmitting, with the one or more electronic processors, the plaque erosion prediction for display via a display device.

In some configurations, the method may include performing, with the one or more electronic processors, momentum distillation-enhanced self-supervised pre-training of the machine learning model prior to receiving the plurality of medical images. In some instances, performing momentum distillation-enhanced self-supervised pre-training of the machine learning model prior to receiving the plurality of medical images may enhance the model performance.

In some configurations, receiving the plurality of medical images may include receiving a plurality of coronary CTA images for a patient that has acute coronary syndromes (ACS).

In some configurations, applying the machine learning model to the plurality of medical images may include: applying, with the one or more electronic processors, a spatial transformer of the machine learning model to each of a plurality of slices included in each of the plurality of medical images to generate a corresponding feature vector for each of the plurality of slices; and applying, with the one or more electronic processors, a sequential transformer of the machine learning model to each corresponding feature vector of each of the plurality of slices to output a transformed feature, where determining the plaque erosion prediction may include determining the plaque erosion prediction based on the transformed feature.

Another configuration may provide a non-transitory, computer-readable medium storing instructions that, when executed by one or more electronic processors, perform a set of functions. The set of functions may include receiving a plurality of CTA images. The set of functions may include applying, to the plurality of CTA images, a modality-specific deep learning model including a spatial transformer and a sequential transformer, where applying the modality-specific deep learning model may include: generating, via the spatial transformer, a corresponding feature vector for each of a plurality of CTA slices included in each of the plurality of CTA images; and outputting, via the sequential transformer, a transformed feature based on each corresponding feature vector of each of the plurality of CTA slices. The set of functions may include determining a plaque erosion prediction based on application of the modality-specific deep learning model to the plurality of CTA images. The set of functions may include transmitting the plaque erosion prediction for display via a display device.

In some configurations, the set of functions may include determining a recommended treatment based on the plaque erosion prediction; and outputting the recommended treatment, where, when the plaque erosion prediction indicates a first probability of plaque erosion, the recommended treatment may include an antithrombotic therapy without stenting, and wherein, when the plaque erosion prediction indicates a second probability of plaque erosion, the recommended treatment may include a percutaneous coronary intervention, where the second probability of plaque erosion is lower than the first probability of plaque erosion.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the technology disclosed herein, however, and reference is therefore made to the claims and herein for interpreting the scope of the technology disclosed herein.

### BRIEF DESCRIPTION OF THE DRAWINGS

The following drawings are provided to help illustrate various features of examples of the disclosure and are not intended to limit the scope of the disclosure or exclude alternative implementations.

FIGS. 1-3 are flowcharts of a patient study in accordance with some configurations disclosed herein.

FIG. 4 is a flowchart illustrating a dataset division for a patient study in accordance with some configurations disclosed herein.

FIG. 5 illustrates an architecture of a CNN model in accordance with some configurations disclosed herein.

FIG. 6 illustrates an architecture of a MD-CTA model, and, in particular, an ability of the MD-CTA model to leverage sequential as well as spatial attention to incorporate the information of the entire series of slices within the coronary CTA scan in accordance with some configurations disclosed herein.

FIG. 7 illustrate a training strategy of a MD-CTA model, and in particular, modality-specific self-supervised pre-training, in accordance with some configurations disclosed herein.

FIG. 8 illustrates a training strategy of a MD-CTA model, and in particular, fine-tuning the training of the MD-CTA model in accordance with some configurations disclosed herein.

FIGS. 9A-9B illustrate patient-level prediction performances, including diagnostic accuracy, for plaque erosion in accordance with some configurations disclosed herein.

FIGS. 10A-10B illustrate slice-level prediction performances in accordance with some configurations disclosed herein.

FIG. 11 illustrates a confusion matrix of a model at a patient-level in accordance with some configurations disclosed herein.

FIG. 12 illustrates a confusion matrix of the model at a slice-level in accordance with some configurations disclosed herein.

FIG. 13 is an OCT image of plaque rupture in accordance with some configurations disclosed herein.

FIG. 14 is a CTA image showing a contrast effect a site of a cavity observed on an OCT image and a vessel lumen in accordance with some configurations disclosed herein.

FIG. 15 is a CTA image showing that a DL model focused on the two contrast effects illustrated in FIG. 14 in accordance with some configurations disclosed herein.

FIG. 16 is an OCT image of plaque erosion in accordance with some configurations disclosed herein.

FIG. 17 is a CTA image showing a small lumen surrounded by plaque without a cavity in accordance with some configurations disclosed herein.

FIG. 18 is a CTA image showing that the DL model concentrated at the site of stenosis without evidence of a cavity in accordance with some configurations disclosed herein.

FIG. 19 is a diagram illustrating automated diagnosis of plaque erosion with a non-invasive coronary CTA using a DL model in accordance with some configurations disclosed herein.

FIG. 20 illustrates an example approach for evaluation and management of patients with ACS in accordance with some configurations disclosed herein.

FIG. 21 illustrates a system for non-invasively diagnosing plaque erosion using coronary CTA with deep learning model(s) according to some configurations.

FIG. 22 illustrates a user device included in the system of FIG. 21 in accordance with some configurations disclosed herein.

FIG. 23 is a flowchart illustrating a method for non-invasively diagnosing plaque erosion using coronary CTA with deep learning model(s) according to some configurations.

### DETAILED DESCRIPTION

### 1—Introduction

As noted herein, acute coronary syndromes (ACS) are the most common cause of death worldwide. Previous reports have revealed that plaque rupture was the underlying mechanism in the majority of cases. However, an alternative pathology, plaque erosion, has been gaining attention as recent in vivo studies demonstrated that erosion is responsible for 25-60% of cases. In current clinical practice, ACS patients are uniformly treated with stenting regardless of the underlying pathology. Recent studies have shown that conservative management without coronary stenting might be an option for ACS patients with plaque erosion.

Currently, a diagnosis of plaque erosion can be made by intracoronary optical coherence tomography (OCT), involves an invasive procedure and expertise in image interpretation. The use of coronary computed tomography angiography (CTA) has been increasing exponentially over the last several years. However, due to limited resolution, coronary CTA has never been evaluated for its ability to make a diagnosis of plaque erosion in which structural changes are subtle.

In recent years, deep learning (DL) has been applied to various medical fields, including medical imaging. Recent works have reported the DL application on automated coronary CTA analyses ranging from segmentation to classification, but the targets for identification were confined to easily discernible findings, such as stenosis or calcification, and the diagnosis of challenging entities, such as plaque erosion, has never been reported. Moreover, explicitly training a model for diagnosis of plaque erosion has never been possible, as the number of patients who underwent coronary CTA paired with concurrent OCT was limited.

Accordingly, the technology disclosed herein aims to develop a DL model to make an accurate diagnosis of plaque erosion non-invasively with coronary CTA. To achieve this aim, the technology disclosed herein may implement the DL model (also referred to herein as the “Momentum Distillation-enhanced Composite Transformer Attention (MD-CTA) model”) that can incorporate information of entire scans, utilizing the modality-specific self-supervised learning strategy to enhance performance.

### 2—Methods

### 2.1—Study Population.

Patients with ACS (non-ST-segment elevation myocardial infarction (NSTEMI) or unstable angina pectoris (UAP)) or stable angina pectoris (SAP) who underwent both coronary CTA and OCT prior to percutaneous coronary intervention (PCI) were included from a database, “Massachusetts General Hospital (Massachusetts, USA) and Tsuchiura Kyodo General Hospital (TKGH) (Ibaraki, Japan) Coronary Imaging Collaboration (NCT04523194). NSTEMI and UAP were diagnosed using American Heart Association/American College of Cardiology guidelines. NSTEMI was defined as ischemic symptoms in the absence of ST-segment elevation on the electrocardiogram with elevated cardiac biomarkers. UAP was defined as having newly developed or accelerating ischemic symptoms on exertion or rest angina within two weeks without biomarker release. SAP was defined as chest pain on exertion without changes in frequency, intensity, and duration of symptoms in the previous four weeks and/or a positive stress test. The culprit lesion was defined as the site of PCI, the tightest lesion, or the lesion with evidence of recent plaque disruption on coronary angiogram. In the case of multivessel PCI, the lesion with the highest degree of stenosis was chosen as the culprit lesion.

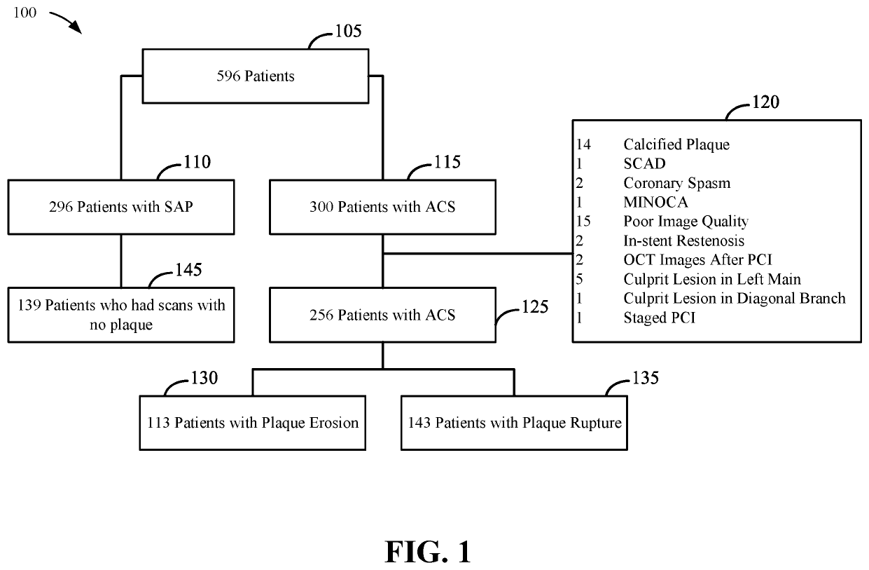

FIG. 1 is a flowchart 100 of the study disclosed herein. As illustrated in FIG. 1, the study included a total of 596 patients (represented in FIG. 1 by reference numeral 105) underwent both CTA and OCT imaging before intervention between January 2011 and July 2020. Of the 596 patients, 296 were patients with SAP (represented in FIG. 1 by reference numeral 110) and 300 were patients with ACS (represented in FIG. 1 by reference numeral 115). Of the 300 patients with ACS 115, a number of patients were excluded (represented in FIG. 1 by reference numeral 120). As illustrated in FIG. 1, the excluded patients included 14 patients for calcified plaque, 1 patient for spontaneous coronary artery dissection, 2 patients for coronary spasm, 1 patient for myocardial infarction with non-obstructive coronary artery, 15 patients for poor image quality, 2 patients for in-stent restenosis, 2 patients for no OCT images before PCI, 5 patients for culprit lesions located in the left main, 1 patient for culprit lesion located in the diagonal branch, and 1 patient for staged PCI. Thus, after removing the excluded patients, the study included 256 Patients with ACS (represented in FIG. 1 by reference numeral 125), including 113 Patients with plaque erosion (represented in FIG. 1 by reference numeral 130) and 143 patients with plaque rupture (represented in FIG. 1 by reference numeral 135). Of the 296 patients with SAP 110, 139 patients who had vessel segments greater than 10 mm in length with no plaque as assessed by angiography and CTA imaging were included in the non-erosion group (represented in FIG. 1 by reference numeral 145). Thus, the 256 Patients with ACS 125, including the 113 patients with plaque erosion 130 and the 143 patients with plaque rupture 135, and 139 patients with SAP 145 were included in the final analysis.

### 2.2—Coronary CTA Acquisition and Analysis.

Coronary CTA image acquisition was performed using a 320-slice CT scanner (Aquilion ONE; Canon Medical Systems Corporation, Otawara, Tochigi, Japan) in accordance with the Society of Cardiovascular Computed Tomography guidelines. Oral and/or intravenous beta-blockers were administered when a patient's resting heart rate was greater than 65 bpm. Sublingual nitroglycerin (0.3 or 0.6 mg) was administered immediately before CT scanning. Coronary CTA images were acquired with the following scan protocol: tube voltage of 120 kVp, tube current of 50 to 750 mA, the gantry rotation speed of 350 ms per rotation, and field matrix of 512×512, and scan slice thickness of 0.5 mm. Acquisition of CT data and the electrocardiography (ECG) trace were automatically started as soon as the signal density level in the ascending aorta reached a predefined threshold of 150 Hounsfield units. Images were acquired after a bolus injection of 30 to 60 mL of contrast media (iopamidol, 370 mg iodine/mL, Bayer Yakuhin, Ltd., Osaka, Japan) at a rate of 3 to 6 mL/s, using prospective ECG-triggering or retrospective ECG-gating with automatic tube current modulation. All scans were performed during a single breath-hold. Images were reconstructed at a window centered at 75% of the R-R interval to coincide with left ventricular diastasis. The coronary CTA datasets were analyzed on a cardiac workstation with dedicated analysis software (QAngio CT RE 3.1, Medis, Leiden, the Netherlands). Analysis began with the automatic detection of the coronary arteries followed by the segmentation of luminal and outer vessel boundaries. If needed, manual adjustments of the vessel centerline and boundaries were performed.

### 2.3—OCT Analysis.

OCT examination was performed using either a frequency-domain (e.g., C7/C8, OCT Intravascular Imaging System, St. Jude Medical, St. Paul, Minnesota) or a time-domain (e.g., M2/M3 Cardiology Imaging Systems, LightLab Imaging Inc., Westford, Massachusetts) OCT system. The images were analyzed by three independent investigators who were blinded to patients' data, using an offline review workstation (St. Jude Medical). Qualitative and quantitative analyses were performed using the previously established criteria.

2.4—Cross-correlation with CTA and OCT Images.

Matching of OCT and CTA images may be performed using an offline model (e.g., Matcher version 2.1 Leiden, the Netherlands). In a first step, OCT images may be mapped onto the CT image along a vessel centerline using anatomical landmarks. In a second step, individual OCT images may be translated and rotated to fit best on the CT image, using, e.g., the vessel center and landmarks for orientation. The offline model may also correct for deviations in the OCT pullback speed by using, e.g., interpolation between landmarks.

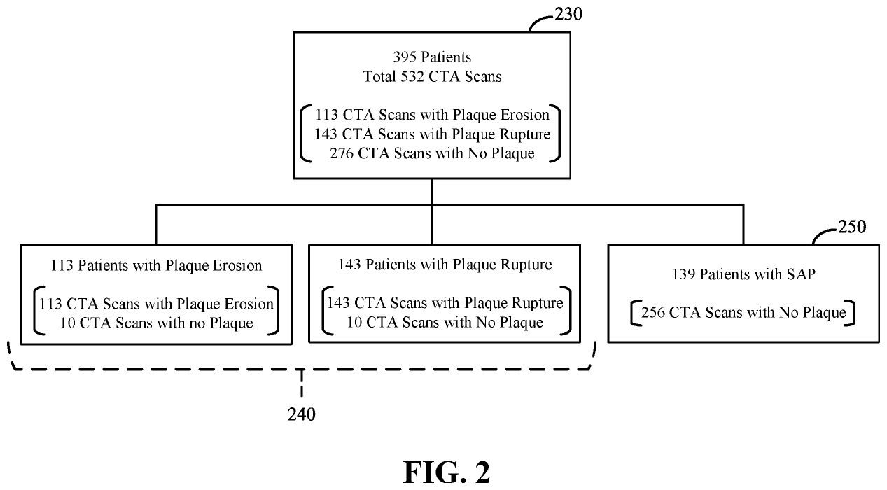

With reference to FIG. 2, among the 256 patients with ACS, the diagnosis of plaque erosion (n=113) or rupture (n=143) on OCT may be used as the ground truth and the site on the CTA image that matched the culprit plaque on the OCT image may be determined to be the culprit lesion. In addition, as illustrated in FIG. 2, 276 CTA scans without plaques detected by OCT and/or angiography and CTA images were chosen as the scans with no plaque. Thus, a total of 532 CTA scans may be included in the final analysis (113 CTA scans with plaque erosion, 143 with plaque rupture, and 276 scans with no plaque) (represented in FIG. 2 by reference numeral 230). Accordingly, as illustrated in FIG. 2, among the 532 CTA scans, 276 CTA scans were from patients with ACS, including 113 CTA scans with plaque erosion, 143 CT scans with plaque rupture, and 20 CT scans with no plaque (represented in FIG. 2 by reference numeral 240), and 256 scans with no plaque were from patients with SAP (represented in FIG. 2 by reference numeral 250).

For the development and validation of the DL model, CTA images in digital imaging and communications in medicine (DICOM) format and corresponding labels of the CTA images were transferred to the Bio-Imaging, Signal Processing, and Learning laboratory at the Korea Advanced Institute of Science and Technology after anonymization.

In deep learning, the entire data corpus is generally divided into subsets: a training dataset, a validation dataset, and a test dataset. The training dataset may be the corpus that the model sees and learns the pattern of data. The training dataset may be used to fit the model parameters, for instance, by training on the data and labels through supervised learning methods using optimization methods, such as, e.g., gradient descent. As training proceeds, the model may eventually become overfitted to the training dataset, meaning that the model may be exorbitantly biased to the pattern of data seen during training (e.g., learned from the training dataset), and, therefore, a generalization performance may be exacerbated. The validation dataset may be used to ameliorate the bias while tuning hyperparameters of the model. The validation dataset may be for the evaluation of a given model but used for frequent evaluation by deep learning developers to fine-tune the model hyperparameters. To further reduce the bias (e.g., as caused by the subset division), k-fold cross-validation (e.g., five-fold cross-validation) may be performed to provide coherent evaluation results for the data used for the model development and tuning. The test dataset, which may be separated prior to the model development and may be used once the model is complete, provides the gold standard for the evaluation of results on an independent set of data. As the test dataset is used to estimate performance of the model in a real-world application, the external dataset collected from an independent institution with different acquisition settings may be used.

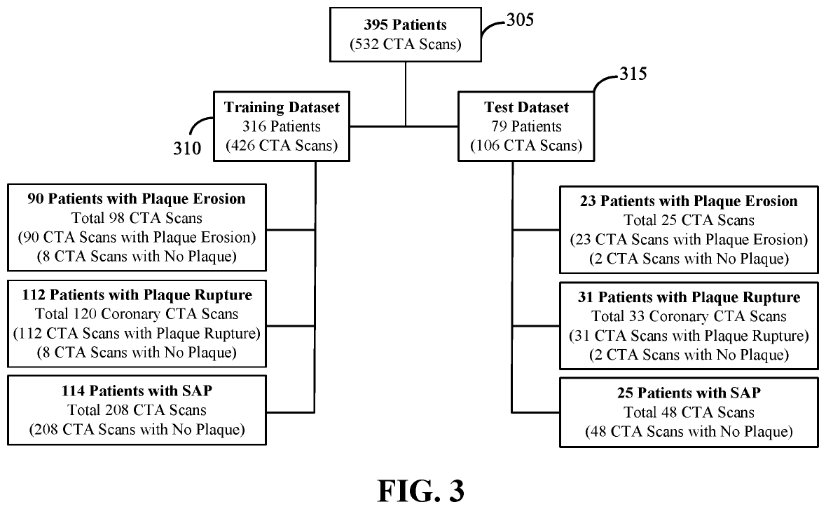

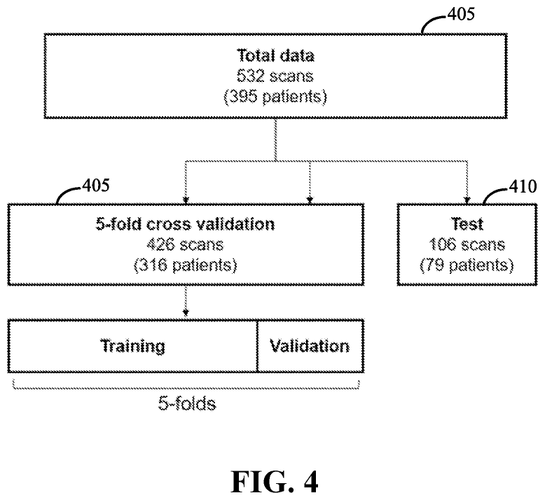

As illustrated in FIG. 3, among 395 patients (532 CTA scans) (represented in FIG. 3 by reference numeral 305), the data was divided into non-overlapping patient subsets, including training and cross-validation datasets containing 316 patients (426 CTA scans) for model development and tuning (represented in FIG. 3 by reference numeral 310), and the test dataset containing 79 patients (106 CTA scans) for final performance evaluation (represented in FIG. 3 by reference numeral 315). As illustrated in FIG. 4, among 532 scans from 395 patients (represented in FIG. 4 by reference numeral 405), the data may be divided into non-overlapping patient subsets. For instance, as illustrated in FIG. 4, 426 scans from 316 patients were used for five-fold cross-validation (represented in FIG. 4 by reference numeral 405), and 106 scans from 79 patients were utilized for the test set validation (represented in FIG. 4 by reference numeral 410).

### 2.5—Development and Evaluation of the Deep Learning Algorithm.

Labels were divided into two classes: plaque erosion and non-plaque erosion. In the non-plaque erosion class, plaque rupture, as well as the other images without significant lesions were included.

To make an accurate diagnosis, the entire collection of CTA images may be into consideration. Thus, a vision transformer (ViT)-based model was design, which was tailored to the data structure of CTA (also referred to herein as “the MD-CTA model”). Unlike most contemporary medical AI models that lack the ability to incorporate the information of the entire volume, the ViT model tailored for sequential data structure may be utilized. Specifically, the spatial transformer that extracts the information within a single slice and the sequence transformer that incorporates the extracted information of all slices to produce the final outcome were simultaneously optimized. The model was trained using both the slice-level and patient-level annotations to enable the model to learn the location of the lesion of interest as well as the label classes. In some configurations, a convolutional neural network (CNN) based model may be implemented for comparison with the same design and settings as the disclosed DL model. A summary of the hyperparameters of the DL model are set forth below in Table 1. An internal five-fold cross-validation may be performed to get the best hyperparameter as well as evaluate the model performance. The model is visualized via the attention weights of the spatial and sequence transformers, as described in greater detail herein.

TABLE 1
Summary of hyperparameters of the deep learning model

| Hyperparameters | Value |
|---|---|
| Input image size | 224 × 224 |
| Batch size | 1 |
| Learning rate | 0.00001 |
| Learning rate scheduler | WarmupCosine |
| Optimizer | AdamW |
| Pre-training epochs | 20 |
| Epochs | 30 |
| Weight decay coefficient | 0.0001 |

Convolutional neural network (CNN) based models may be used as a standard model architecture for medical images as well as computer vision, due to performance with the convolution operation. Transformer-based models generally provide scaling properties and powerful self-attention mechanisms, and, as such, have generally overtaken the CNN-based models. For example, vision Transformer (ViT), relying on pure self-attention, surpassed the state-of-the-art CNN-based model without using the convolution operation. ViT provides advantageous scalability that can benefit more from large model and dataset sizes. ViT may include useful properties desired in the domain of medical imaging. ViT has the shape-biased property, such that the ViT model makes a decision, concentrating more on a shape of an object rather than background noise or other confounders. Moreover, a well-trained ViT model has a flatter loss landscape compared to the CNN-based model, allowing generalizability for many tasks. Finally, the knowledge distillation-based self-supervised learning strategy, an autodidactic learning approach where the student model learns from the teacher model's prediction in place of the label, may be especially effective for the ViT models.

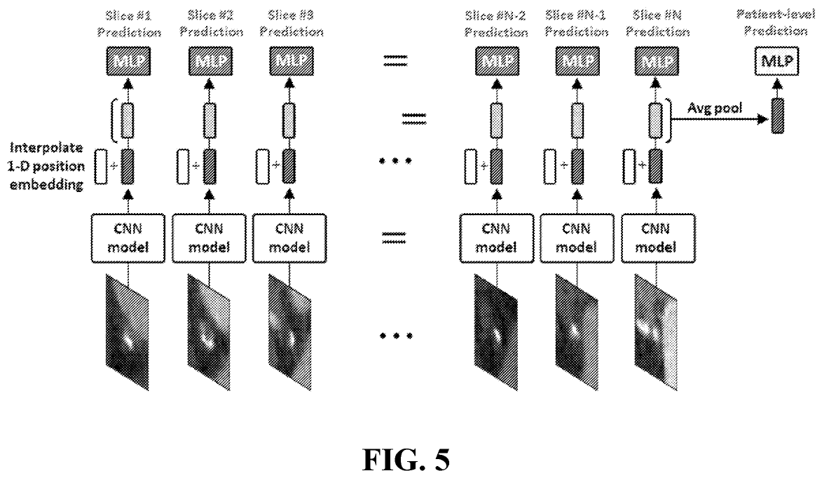

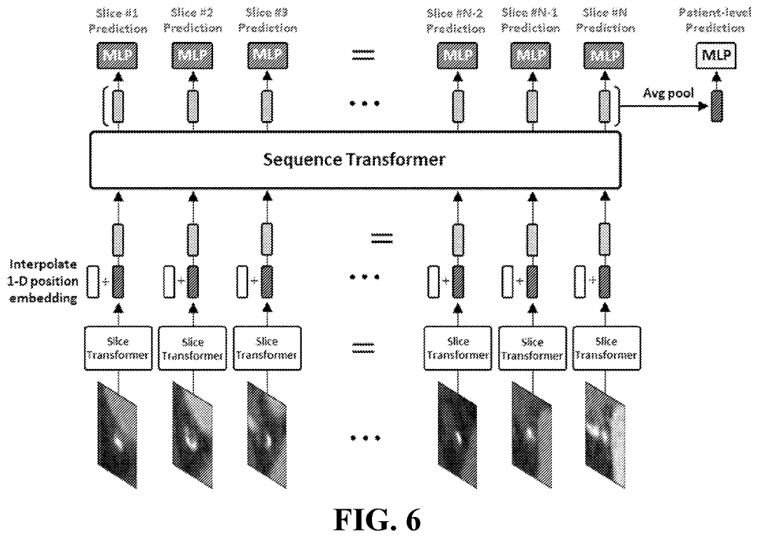

In some configurations, the technology disclosed herein may implement (or otherwise provide) a ViT-based DL model for coronary CTA image processing and analysis (e.g., the MD-CTA model). In some configurations, the MD-CTA model disclosed herein may include a composite transformer attention component, a momentum distillation component, or a combination thereof. The composite transformer attention component may implement transformer attention in two ways. As one example, a pure ViT model equipped with the self-attention within each slice (e.g., a spatial transformer), may be used to encode the CTA slices into a more compact feature vector, such as, e.g., with a dimension of 384. A sequential transformer may subsequently receive (or otherwise access) the feature vectors of all slices and output a transformed feature incorporating the information of the entire CTA scans. Slice-level and the patient-level prediction results may be obtained from the transformed feature with a simple multi-layer perception and an average pooling operation, respectively. Multi-task learning may improve performance of deep learning models. As such, the technology disclosed herein may simultaneously optimize the model with two learning objectives from two different tasks, such as, e.g., the slice-level prediction and the patient-level prediction, as illustrated in FIGS. 5-6.

FIG. 5 illustrates an architecture of a CNN model. FIG. 6 illustrates an architecture of the MD-CTA model, and, in particular, an ability of the MD-CTA model to leverage sequential as well as spatial attention to incorporate the information of the entire series of slices within the coronary CTA scan, which is not possible for the CNN model of FIG. 5.

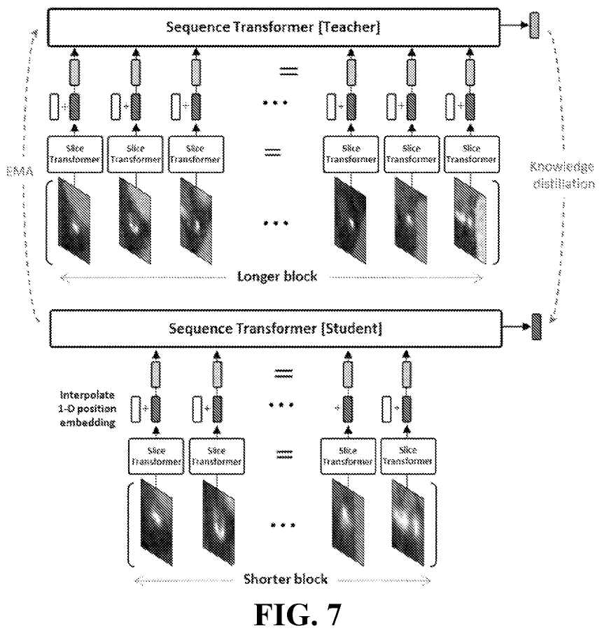

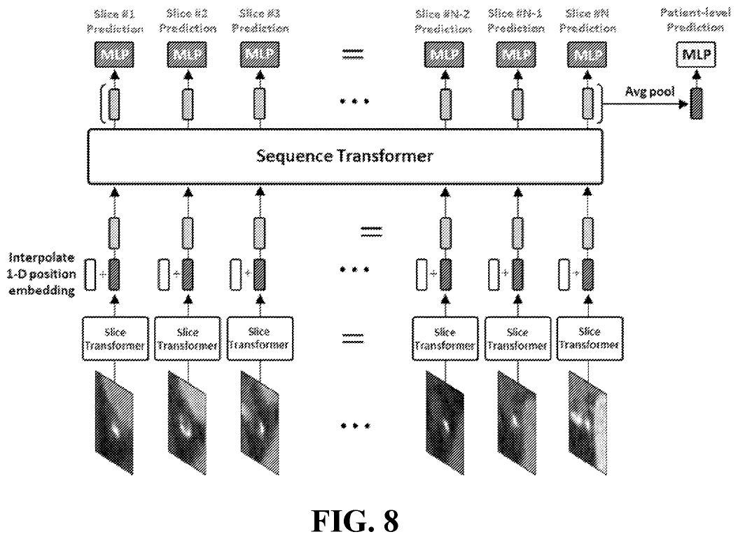

Performance of the model may be improved with the momentum distillation-enhanced self-supervised pre-training, as illustrated in FIGS. 7-8. FIG. 7 illustrates a training strategy of the MD-CTA model, and in particular, modality-specific self-supervised pre-training. FIG. 8 illustrates a training strategy of the MD-CTA model, and in particular, fine-tuning the training of the MD-CTA model. For instance, in some cases, as illustrated in FIG. 7, two identical models as teacher and student may be implemented, and the CTA volume may be cropped into a longer and smaller sequence. Then, the longer sequence may be input to the teacher, while the shorter sequence may be input to the student. By letting the student model match the prediction of the teacher model with less information (shorter sequence), the model can learn the knowledge about the imaging modality without any handcrafted supervision. Accordingly, in some instances, the MD-CTA model may be first pre-trained with the momentum distillation-enhanced self-supervised learning (as illustrated in FIG. 7), and subsequently fine-tuned to optimize patient-level and slice-level predictions simultaneously (as illustrated in FIG. 8). Such a step-wise strategy may significantly improve the overall performance of the model.

In some configurations, the spatial transformer may include a ViT-B/16 model pre-trained on ImageNet, and the transformer may be equipped with 12 layers and 12 attention heads as the sequential encoder. Considering the complexity of the model, a ResNet-50 model may be used as the CNN model for comparison.

A reader study was performed to evaluate the clinical utility of the DL model as an assisting tool as well as to compare the performances with experienced cardiologists. To this aim, the reader study may be performed twice. In the first round, the performance of the DL model for the test set was compared with that of cardiologists who had more than 8 years of experience. In particular, the anonymized 106 CTA scans in the test set were given to three cardiologists along with an answer sheet to complete. The cardiologists were blinded to clinical information and OCT findings for a fair comparison with the model. The performance comparison was conducted by comparing their performances at this time with the model's performances. Then, in the second round, the prediction results by the DL model along with the corresponding CTA scans were provided to the readers to evaluate whether the diagnostic performances were improved with the model's assistance. In the second round, the test set was randomly shuffled again and given to the readers along with the model's prediction, after a four-week washout period to prevent the performance improvement from the recollection. In this round, we aimed to investigate whether giving the model's prediction for a given CTA scan could improve the sensitivity, specificity, and accuracy of the experienced cardiologist's coronary CTA reading.

### 2.6—Statistical Analysis.

Categorical data are presented as counts and percentages, and are compared using the chi-squared test or Fisher exact test, as appropriate. Continuous variables have been shown as mean±SD or median (25th to 75th percentiles), as appropriate, depending on the normality of distribution. Per-lesion data were analyzed using the generalized estimating equations with a logit link for the binary variables to consider the potential clustering of multiple plaques in a single patient. Between-group differences in continuous variables were compared using the Student t-test or Mann-Whitney U test, as appropriate. A P value <0.05 was considered statistically significant.

The model performance was evaluated with the area under the receiver-operating-characteristic curves (AUC), and the sensitivities, specificities, accuracy, positive predictive values (PPVs), and negative predictive values (NPVs) were calculated for the detailed analysis. To estimate the false alarms by the model, the false-positive rate (FPR) and false-negative rate (FNR) were calculated. The 95% confidence intervals (CIs) were calculated by DeLong's method for AUC, and “exact” Clopper-Pearson confidence intervals for sensitivity, specificity, accuracy, and false estimates. Likewise, the standard logit confidence intervals were used to estimate the 95% CIs of the predictive values.

### 3—Results

### 3.1—Study Population.

For model development and internal validation, a total of 532 CTA scans from 395 patients were used. Patients were randomly divided into non-overlapping subsets, training and cross-validation datasets for model development and tuning (426 scans from 316 patients), and the test set for final performance evaluation (containing 106 scans from 79 patients), as illustrated in FIG. 3.

Detailed patient characteristics are summarized in Table 2 (below).

TABLE 2
Patient characteristics in the training versus test datasets

| | | Overall Patients (n = 395) | | |
|---|---|---|---|---|
| | | Training Dataset | Test Dataset | |
| Variables | | (n = 316, 80.0%) | (n = 79, 20.0%) | P Value |
| Age (years) | | 66.5 (58.3-74.0) | 68.5 (59.0-76.0) | 0.893 |
| Male | | 257 (81.3) | 66 (83.5) | 0.648 |
| Hypertension | | 188 (59.5) | 48 (60.8) | 0.690 |
| Dyslipidemia | | 135 (42.7) | 40 (50.6) | 0.371 |
| Diabetes mellitus | | 109 (34.5) | 18 (22.8) | 0.018 |
| Current smoking | | 93 (29.4) | 24 (30.4) | 0.951 |
| Renal insufficiency | | 102 (32.3) | 22 (27.8) | 0.448 |
| Ejection fraction (%) | | 63 (56-67) | 64 (55-68) | 0.618 |
| NSTE-ACS | NSTEMI | 161 (50.9) | 37 (46.8) | 0.513 |
| | UAP | 41 (13.0) | 17 (21.5) | 0.055 |

| | | | |
|---|---|---|---|
| SAP | 114 (36.1) | 25 (31.6) | 0.461 |
| Previous MI | 40 (12.7) | 6 (7.6) | 0.210 |
| Previous PCI | 49 (15.5) | 10 (12.7) | 0.519 |
| Previous CABG | 4 (1.3) | 0 (0.0) | 0.315 |
| Medication on admission | | | |
| Aspirin | 85 (26.9) | 22 (27.8) | 0.609 |
| DAPT | 60 (19.0) | 16 (20.3) | 0.782 |
| ACE-I/ARB | 100 (31.6) | 33 (41.8) | 0.160 |
| Statin | 111 (35.1) | 25 (31.6) | 0.348 |
| B-blocker | 130 (41.1) | 35 (44.3) | 0.902 |
| Laboratory data | | | |
| WBC (count/μL) | 6660 (5380-8738) | 6965 (5565-8735) | 0.901 |
| Triglycerides (mg/dL) | 114 (82-183) | 124 (84-197) | 0.399 |
| Total cholesterol (mg/dL) | 185 ± 2.6 | 192 ± 5.7 | 0.450 |
| LDL cholesterol (mg/dL) | 111 (88-137) | 117 (92-138) | 0.671 |
| HDL cholesterol (mg/dL) | 46 (40-56) | 46 (41-56) | 0.944 |
| Glucose (mg/dL) | 120 (101-154) | 119 (104-139) | 0.506 |
| HbA1c (%) | 5.9 (5.5-6.8) | 5.9 (5.5-6.6) | 0.355 |
| eGFR (mL/min/1.73 m²) | 72.9 (61.2-83.3) | 72.5 (62.9-84.4) | 0.212 |

Where, values are mean±SD, n (%), or median (25^{th}-75^{th} percentile). As used herein, “ACE-I” represents angiotensin-converting enzyme inhibitor; “ARB” represents angiotensin II receptor blocker; “CABG” represents coronary artery bypass graft; “DAPT” represents dual anti-platelet therapy; “eGFR” represents estimated glomerular filtration rate; “HibAlc” represents hemoglobin Alc; “HIDL” represents high-density lipoprotein; “LDL” represents low-density lipoprotein; “MI” represents myocardial infarction; “NSTE-ACS” represents non-ST-segment elevation acute coronary syndromes; “NSTEMI” represents non-ST-segment elevation myocardial infarction; “PCI” represents percutaneous coronary intervention: “SAP” represents stable angina pectoris; “UAP” represents unstable angina pectoris; and “WBC” represents white blood cell.

Other than a higher prevalence of diabetes mellitus in the training dataset than in the test dataset (109 [34.5%] vs. 18 [22.8%], p=0.018), no differences were observed in patient characteristics, medications, and laboratory data between the two datasets. The subset of patients with ACS showed the same pattern (Diabetes: 78 [38.6%] vs. 11 [20.4%], p=0.013), as set forth in Table 3 (below).

TABLE 3
Comparison of baseline characteristics in ACS patients between training and test datasets

| | ACS Overall patients (n = 256) | | |
|---|---|---|---|
| | Training Dataset | Test Dataset | |
| Variables | (n = 202, 78.9%) | (n = 54, 21.1%) | P Value |
| Age (years) | 64.5 (54.3-71.8) | 60.0 (52.0-71.0) | 0.891 |
| Male | 164 (81.2) | 45 (83.3) | 0.718 |
| Hypertension | 134 (66.3) | 35 (64.8) | 0.858 |
| Dyslipidemia | 97 (48.0) | 25 (46.3) | 0.839 |
| Diabetes mellitus | 78 (38.6) | 11 (20.4) | 0.013 |
| Current smoking | 75 (37.1) | 19 (35.2) | 0.879 |
| Renal insufficiency | 43 (21.3) | 12 (22.2) | 0.882 |
| Ejection fraction (%) | 61 (52-65) | 63 (49-65) | 0.749 |
| NSTEMI | 161 (79.7) | 37 (68.5) | 0.081 |
| UAP | 41 (20.3) | 17 (31.5) | 0.081 |
| Previous MI | 16 (7.9) | 3 (5.6) | 0.556 |
| Previous PCI | 17 (8.4) | 4 (7.4) | 0.810 |
| Previous CABG | 4 (2.0) | 0 (0.0) | 0.297 |
| Medication on admission | | | |
| Aspirin | 36 (17.8) | 9 (16.7) | 0.700 |
| DAPT | 16 (7.9) | 3 (5.6) | 0.486 |
| ACE-I/ARB | 84 (41.6) | 29 (53.7) | 0.104 |
| Statin | 56 (27.7) | 10 (18.5) | 0.172 |
| B-blocker | 98 (48.5) | 28 (51.9) | 0.643 |
| Laboratory data | | | |
| WBC (count/μL) | 7370 (5918-9835) | 8140 (6375-10020) | 0.919 |
| Triglycerides | 119 (73-167) | 163 (72-202) | 0.420 |
| (mg/dL) | | | |
| Total cholesterol | 193 ± 5.9 | 204 ± 13.3 | 0.250 |
| (mg/dL) | | | |
| LDL cholesterol | 122 ± 5.1 | 126 ± 11.5 | 0.485 |
| (mg/dL) | | | |
| HDL cholesterol | 49 ± 1.6 | 49 ± 3.3 | 0.533 |
| (mg/dL) | | | |
| Glucose (mg/dL) | 116 (100-153) | 113 (107-131) | 0.286 |
| HbA1c (%) | 5.8 (5.4-6.6) | 5.6 (5.3-5.8) | 0.127 |
| eGFR | 75.9 (66.3-84.9) | 80.3 (72.5-88.7) | 0.418 |
| (mL/min/1.73 m²) | | | |
| Lesion location | | | 0.797 |
| RCA | 50 (24.8) | 14 (25.9) | |
| LAD | 110 (54.5) | 31 (57.4) | |
| LCX | 42 (20.8) | 9 (16.7) | |
| Pathology | | | |
| Plaque erosion | 90 (44.6) | 23 (42.6) | 0.796 |
| Plaque rupture | 112 (55.4) | 31 (57.4) | 0.796 |
| OCT findings | | | |
| Lipid-rich plaque | 171 (84.7) | 48 (88.9) | 0.432 |
| TCFA | 84 (41.6) | 24 (44.4) | 0.705 |
| Macrophage | 158 (78.2) | 48 (88.9) | 0.079 |
| Microvessels | 96 (47.5) | 25 (46.3) | 0.872 |
| Cholesterol crystal | 58 (28.7) | 14 (25.9) | 0.686 |
| Layered plaque | 101 (50.0) | 26 (48.1) | 0.809 |
| Calcifications | 98 (48.5) | 24 (44.4) | 0.595 |
| Thrombus | 136 (67.3) | 37 (68.5) | 0.868 |
| Minimum fibro cap | 70 (60-110) | 63 (51.5-106.5) | 0.736 |
| thickness (μm) | | | |
| Minimum lumen | 1.01 (0.69-1.18) | 1.05 (0.77-1.81) | 0.462 |
| area (mm²) | | | |
| Area stenosis (%) | 82.7 (75.7-87.2) | 78.7 (69.8-81.7) | 0.663 |
| Plaque length | 11.7(9.2-15.0) | 12.2 (11.3-15.2) | 0.199 |
| (mm) | | | |
| Max lipid arc (°) | 239.3 (203.5-306.6) | 226.7 (185.8-311.5) | 0.811 |
| Mean lipid arc (°) | 203.6 ± 6.5 | 201.5 ± 12.8 | 0.924 |
| Lipid index (°mm) | 1557.7 (996.6-2570.3) | 1460.1 (1181.1-2211.0) | 0.324 |

where, values are mean ± SD, n (%), or median (25^{th}-75^{th} percentile).

As used herein, “ACS” represents acute coronary syndromes; “LAD” represents Left anterior descending artery; “LCX” represents Left circumflex artery; “RCA” represents right coronary artery; and “TCFA” represents thin-cap fibroatheroma.

When the location of the culprit lesion and underlying pathology were compared between training and test datasets, no significant difference was found between the two groups, as set forth in Table 4 (below), where values are n (%).

TABLE 4
Coronary CTA scan lesion characteristics
in the training versus test datasets

| | Overall CTA scans (n = 532) | | |
|---|---|---|---|
| | CTA scans in | CTA scans in | |
| | Training Dataset | Test Dataset | |
| Variables | (n = 426, 80.1%) | (n = 106, 19.9%) | P Value |
| Lesion location | | | 0.762 |
| RCA | 135 (31.7) | 37 (34.9) | |
| LAD | 184 (43.2) | 43 (40.6) | |
| LCX | 107 (25.1) | 26 (24.5) | |
| Pathology | | | |
| Plaque erosion | 90 (21.1) | 23 (21.7) | 0.904 |
| Plaque rupture | 112 (26.3) | 31 (29.2) | 0.577 |
| No plaque | 224 (52.6) | 52 (49.1) | 0.598 |

### 3.2—Diagnostic Performances of the Deep Learning Model.

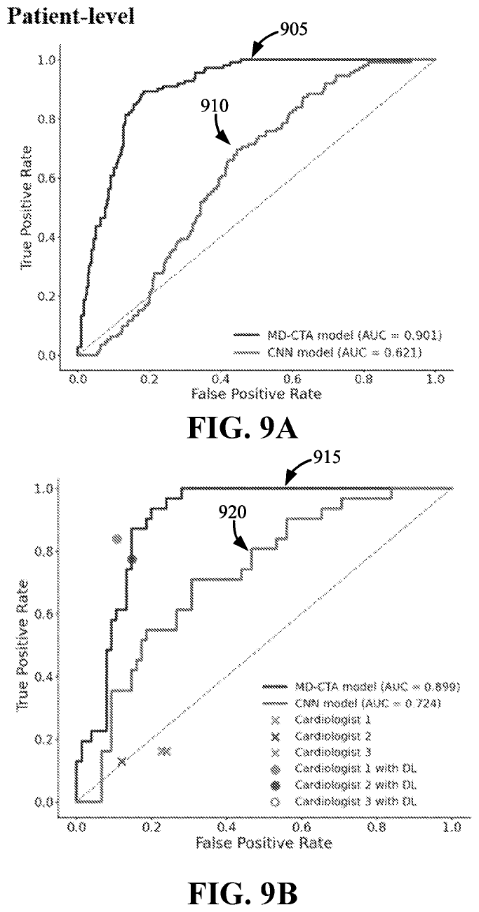

Patient-level prediction performances, including diagnostic accuracy, for plaque erosion are illustrated in FIGS. 9A-91B. FIG. 9A is a graph 900 illustrating diagnostic performance of the DL model (represented in FIG. 9A by reference numeral 905) and a CNN model (represented in FIG. 9A by reference numeral 910) at the patient level in the five-fold cross-validation. FIG. 9B is a graph illustrating diagnostic performance of the DL model (represented in FIG. 9B by reference numeral 915) and a CNN model (represented in FIG. 9B by reference numeral 920) at the patient level in the test set validation. Performance of the DL model for patient-level diagnosis is further set forth in Table 5 (below).

TABLE 5
Performances of the deep learning models for patient-level diagnosis

| | | | | | | |
|---|---|---|---|---|---|---|
| | Sensitivity | Specificity | | | FPR | FNR |
| AUC | (%) | (%) | PPV | NPV | (%) | (%) |
| (95% CI) | (95% CI) | (95% CI) | (95% CI) | (95% CI) | (95% CI) | (95% CI) |

Five-fold cross-validation

| | | | | | | | |
|---|---|---|---|---|---|---|---|
| MD-CTA | 0.901 | 81.2 | 86.6 | 68.4 | 92.8 | 13.4 | 18.8 |
| model | (0.873-0.930) | (72.8-88.0) | (82.4-90.2) | (61.7-74.4) | (89.8-95.0) | (9.8-17.6) | (12.0-27.2) |
| CNN | 0.621 | 59.8 | 60.2 | 34.9 | 80.8 | 39.8 | 40.2 |
| model | (0.567-0.675) | (50.1-69.0) | (54.5-65.7) | (30.4-39.7) | (76.7-84.3) | (34.3-45.5) | (31.0-49.9) |

Test set validation

| | | | | | | | |
|---|---|---|---|---|---|---|---|
| MD-CTA | 0.899 | 87.1 | 85.3 | 71.1 | 94.1 | 14.7 | 12.9 |
| model | (0.841-0.957) | (70.2-96.4) | (75.3-92.4) | (58.3-81.2) | (86.5-97.6) | (7.6-24.7) | (3.6-29.8) |
| CNN | 0.724 | 71.0 | 68.0 | 47.8 | 85.0 | 32.0 | 29.0 |
| model | (0.622-0.826) | (52.0-85.8) | (56.2-78.3) | (38.1-57.8) | (76.2-90.9) | (21.7-43.8) | (14.2-48.0) |

As illustrated in Table 5 and FIG. 9A, in the five-fold cross-validation, the MD-CTA model showed diagnostic performance with an AUC of 0.901 (0.873-0.930), sensitivity of 81.2 (72.8-88.0), and specificity of 86.6 (82.4-90.2), all of which were significantly higher than those of the CNN model with an AUC of 0.621 (0.567-0.675), sensitivity of 59.8 (50.1-69.0), and specificity of 60.2 (54.5-65.7). Similarly, as illustrated in Table 5 and FIG. 9B, in the test set validation, the AUC, sensitivity, and specificity of the DL model were 0.899 (0.841-0.957), 87.1 (70.2-96.4), and 85.3 (75.3-92.4), respectively, higher than those of 0.724 (0.622-0.826), 71.0 (52.0-85.8), and 68.0 (56.2-78.3) of the CNN model. In both five-fold cross-validation and test set validation, the NPVs were higher than 900% but PPVs were relatively low (68.4 and 71.1, respectively) due to the smaller number of positives.

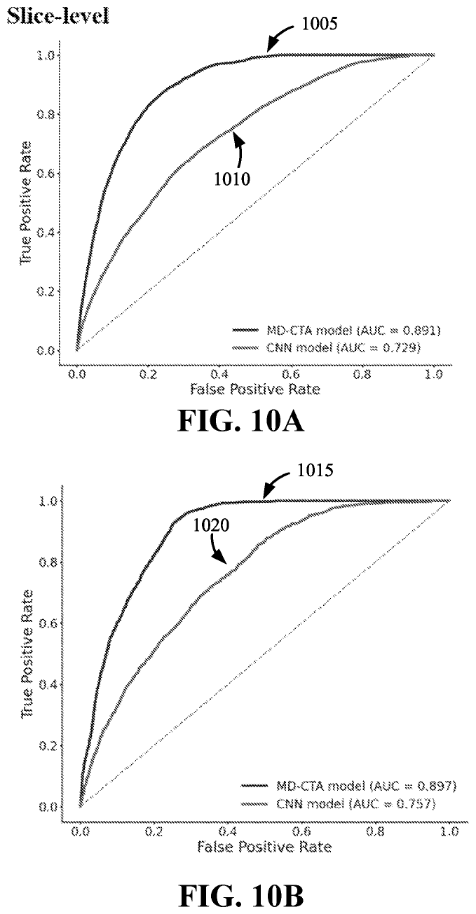

Slice-level prediction performances are illustrated in FIGS. 10A-10B. FIG. 10A is a graph illustrating diagnostic performances of the DL model (represented in FIG. 10A by reference numeral 1005) and a CNN model (represented in FIG. 10A by reference numeral 1010) at the slice level in the five-fold cross-validation. FIG. 10B is a graph illustrating diagnostic performances of the DL model (represented in FIG. 10B by reference numeral 1015) and a CNN model (represented in FIG. 10B by reference numeral 1020) at the slice level in the test set validation. Performance of the DL model for slice-level diagnosis is further set forth in Table 6 (below).

TABLE 6
Performances of the deep learning models for slice-level diagnosis

| | | | | | | |
|---|---|---|---|---|---|---|
| | Sensitivity | Specificity | | | FPR | FNR |
| AUC | (%) | (%) | PPV | NPV | (%) | (%) |
| (95% CI) | (95% CI) | (95% CI) | (95% CI) | (95% CI) | (95% CI) | (95% CI) |

Five-fold cross-validation

| | | | | | | | |
|---|---|---|---|---|---|---|---|
| MD-CTA | 0.891 | 82.9 | 80.0 | 38.5 | 96.9 | 20.0 | 17.1 |
| model | (0.887-0.895) | (81.7-84.0) | (79.5-80.5) | (37.9-39.1) | (96.7-97.1) | (19.5-20.5) | (16.0-18.3) |
| CNN | 0.729 | 66.2 | 66.8 | 23.2 | 92.9 | 33.2 | 33.8 |
| model | (0.722-0.737) | (64.8-67.6) | (66.3-67.4) | (22.7-23.7) | (92.6-93.2) | (32.6-33.7) | (32.4-35.2) |

Test set validation

| | | | | | | | |
|---|---|---|---|---|---|---|---|
| MD-CTA | 0.897 | 82.2 | 80.1 | 39.3 | 96.6 | 19.9 | 17.8 |
| model | (0.890-0.904) | (79.8-84.3) | (79.1-81.0) | (38.0-40.5) | (96.2-97.0) | (19.0-80.9) | (15.7-20.2) |
| CNN | 0.757 | 68.9 | 67.3 | 24.9 | 93.3 | 32.7 | 31.1 |
| model | (0.744-0.770) | (66.2-71.6) | (66.3-68.4) | (23.9-25.8) | (92.7-93.8) | (31.6-33.7) | (28.4-33.8) |

As illustrated in Table 6 and FIG. 10A, in the five-fold cross-validation, the MD-CTA model provided the diagnostic performances with an AUC of 0.891 (0.887-0.895), sensitivity of 82.9 (81.7-84.0), and specificity of 80.0 (79.5-80.5), while the CNN model showed an AUC of 0.729 (0.722-0.737), sensitivity of 66.2 (64.8-67.6), and specificity of 66.8 (66.3-67.4). As illustrated in Table 6 and FIG. 10B, in the test set validation, the MID-CTA model's AUC, sensitivity, specificity, and accuracy were 0.897 (0.890-0.904), 82.2 (79.8-84.3), and 80.1 (79.1-81.0), while those of the CNN model were 0.757 (0.744-0.770), 68.9 (66.2-71.6), and 67.3 (66.3-68.4), respectively. The NPVs for the slice level prediction were over 90.0% in both five-fold cross-validation and the test set validation, while the PPVs were relatively low, attributed to the imbalance between positives and negatives.

Table 7 (below) shows the results of the ablation studies for the composite transformer attention and a modality-specific self-supervised pre-training components of the MD-CTA model. Compared with a ViT model with only the spatial attention or a ViT model with sequential attention to integrate the information of the entire slices to make a decision, the MD-CTA model disclosed herein as adopting the modality-specific self-supervised pre-training with momentum distillation provided overall better performance in both patient-level and slice-level diagnosis.

TABLE 7
Ablation studies on the key components of the Momentum Distillation-
enhanced Composite Transformer Attention (MD-CTA) model

| | | | | | | |
|---|---|---|---|---|---|---|
| | Sensitivity | Specificity | | | FPR | FNR |
| AUC | (%) | (%) | PPV | NPV | (%) | (%) |
| (95% CI) | (95% CI) | (95% CI) | (95% CI) | (95% CI) | (95% CI) | (95% CI) |

Patient-level diagnosis

| | | | | | | | |
|---|---|---|---|---|---|---|---|
| ViT | 0.884 | 80.7 | 77.3 | 59.5 | 90.6 | 22.7 | 19.3 |
| | (0.823-0.945) | (62.5-92.6) | (66.2-86.2) | (48.3-69.8) | (82.3-95.3) | (13.8-33.8) | (7.4-37.5) |
| ViT + ST | 0.872 | 80.7 | 78.7 | 61.0 | 90.8 | 21.3 | 19.3 |
| | (0.807-0.937) | (62.5-92.6) | (67.7-87.3) | (49.5-71.4) | (82.6-95.3) | (12.7-32.3) | (7.4-37.5) |
| ViT + ST + MD | 0.899 | 87.1 | 85.3 | 71.1 | 94.1 | 14.7 | 12.9 |
| (MD-CTA) | (0.841-0.957) | (70.2-96.4) | (75.3-92.4) | (58.3-81.2) | (86.5-97.6) | (7.6-24.7) | (3.6-29.8) |

Slice-level diagnosis

| | | | | | | | |
|---|---|---|---|---|---|---|---|
| ViT | 0.886 | 76.0 | 80.8 | 38.2 | 95.6 | 19.2 | 24.0 |
| | (0.878-0.894) | (73.4-78.4) | (79.8-81.7) | (36.9-39.6) | (95.1-96.0) | (18.2-20.2) | (21.6-26.6) |
| ViT + ST | 0.902 | 81.0 | 82.4 | 41.9 | 96.5 | 17.6 | 19.0 |
| | (0.895-0.909) | (78.6-83.3) | (81.5-83.2) | (40.5-43.3) | (96.1-96.9) | (16.8-18.5) | (16.7-21.4) |
| ViT + ST + MD | 0.897 | 82.2 | 80.1 | 39.3 | 96.6 | 19.9 | 17.8 |
| (MD-CTA) | (0.890-0.904) | (79.8-84.3) | (79.1-81.0) | (38.0-40.5) | (96.2-97.0) | (19.0-20.9) | (15.7-20.2) |

3.3—Analysis of/the False Estimates.

Tables 8 and 9 (below) illustrate the results of an analysis of the false estimates.

TABLE 8
Performances of the deep learning models for patient-level diagnosis

| | | | | | | |
|---|---|---|---|---|---|---|
| | Sensitivity | Specificity | | | FPR | FNR |
| AUC | (%) | (%) | PPV | NPV | (%) | (%) |
| (95% CI) | (95% CI) | (95% CI) | (95% CI) | (95% CI) | (95% CI) | (95% CI) |

Five-fold cross-validation

| | | | | | | | |
|---|---|---|---|---|---|---|---|
| MD-CTA | 0.901 | 81.2 | 86.6 | 68.4 | 92.8 | 13.4 | 18.8 |
| model | (0.873-0.930) | (72.8-88.0) | (82.4-90.2) | (61.7-74.4) | (89.8-95.0) | (9.8-17.6) | (12.0-27.2) |
| CNN | 0.621 | 59.8 | 60.2 | 34.9 | 80.8 | 39.8 | 40.2 |
| model | (0.567-0.675) | (50.1-69.0) | (54.5-65.7) | (30.4-39.7) | (76.7-84.3) | (34.3-45.5) | (31.0-49.9) |

Test set validation

| | | | | | | | |
|---|---|---|---|---|---|---|---|
| MD-CTA | 0.899 | 87.1 | 85.3 | 71.1 | 94.1 | 14.7 | 12.9 |
| model | (0.841-0.957) | (70.2-96.4) | (75.3-92.4) | (58.3-81.2) | (86.5-97.6) | (7.6-24.7) | (3.6-29.8) |
| CNN | 0.724 | 71.0 | 68.0 | 47.8 | 85.0 | 32.0 | 29.0 |
| model | (0.622-0.826) | (52.0-85.8) | (56.2-78.3) | (38.1-57.8) | (76.2-90.9) | (21.7-43.8) | (14.2-48.0) |

TABLE 9
Performances of the deep learning models for slice-level diagnosis

| | | | | | | |
|---|---|---|---|---|---|---|
| | Sensitivity | Specificity | | | FPR | FNR |
| AUC | (%) | (%) | PPV | NPV | (%) | (%) |
| (95% CI) | (95% CI) | (95% CI) | (95% CI) | (95% CI) | (95% CI) | (95% CI) |

Five-fold cross-validation

| | | | | | | | |
|---|---|---|---|---|---|---|---|
| MD-CTA | 0.891 | 82.9 | 80.0 | 38.5 | 96.9 | 20.0 | 17.1 |
| model | (0.887-0.895) | (81.7-84.0) | (79.5-80.5) | (37.9-39.1) | (96.7-97.1) | (19.5-20.5) | (16.0-18.3) |
| CNN | 0.729 | 66.2 | 66.8 | 23.2 | 92.9 | 33.2 | 33.8 |
| model | (0.722-0.737) | (64.8-67.6) | (66.3-67.4) | (22.7-23.7) | (92.6-93.2) | (32.6-33.7) | (32.4-35.2) |

Test set validation

| | | | | | | | |
|---|---|---|---|---|---|---|---|
| MD-CTA | 0.897 | 82.2 | 80.1 | 39.3 | 96.6 | 19.9 | 17.8 |
| model | (0.890-0.904) | (79.8-84.3) | (79.1-81.0) | (38.0-40.5) | (96.2-97.0) | (19.0-80.9) | (15.7-20.2) |
| CNN | 0.757 | 68.9 | 67.3 | 24.9 | 93.3 | 32.7 | 31.1 |
| model | (0.744-0.770) | (66.2-71.6) | (66.3-68.4) | (23.9-25.8) | (92.7-93.8) | (31.6-33.7) | (28.4-33.8) |

As illustrated in Tables 8-9, in the five-fold cross-validation, the FPR and FNR of the MD-CTA model were 13.4 (9.8-17.6) and 18.8 (12.0-27.2) for the patient-level diagnosis, and 20.0 (19.5-20.5) and 17.1 (16.0-18.3) for the slice-level diagnosis, which was lower than the CNN model. In the test set validation, the FPR and FNR of the MD-CTA model were 14.7 (7.6-24.7) and 12.9 (3.6-29.8) for the patient-level, and 19.9 (19.0-80.9) and 17.8 (15.7-20.2) for the slice-level diagnoses, providing lower false estimates than the CNN model.

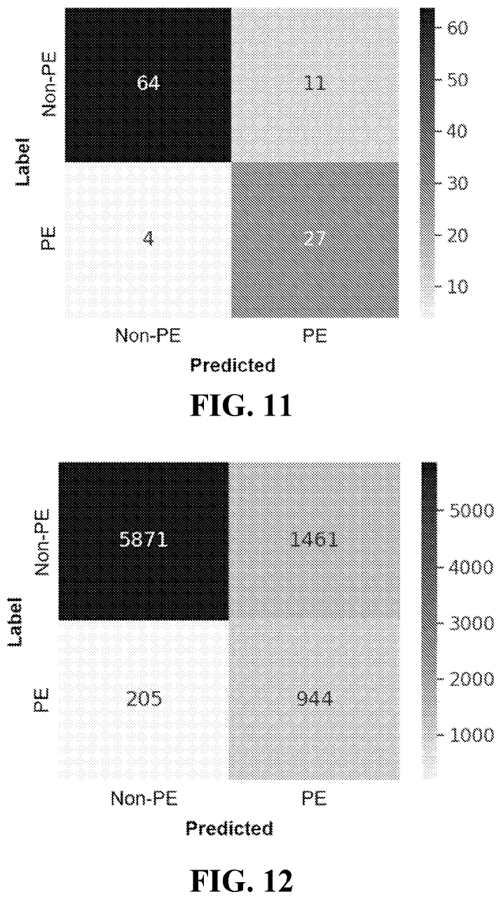

For instance, all 35 cases with false positives were attributed to missed small ruptures and mild irregularity of the lumen contour. In 11 cases of false positives, there was severe calcification in two cases and the other nine cases had relatively severe stenosis. Out of four cases of false negatives, a large red thrombus was misdiagnosed as plaque rupture in two cases, a side branch was misdiagnosed in one case, while a small thrombus attached to the lumen was missed by the DL model in one case. For example, FIGS. 11-12 illustrate confusion matrices of the model. FIG. 11 illustrates a confusion matrix of the model at the patient-level and FIG. 12 illustrates a confusion matrix of the model at the slice-level. As used herein, “PE” represents plaque erosion.

### 3.4—Model Interpretability Results.

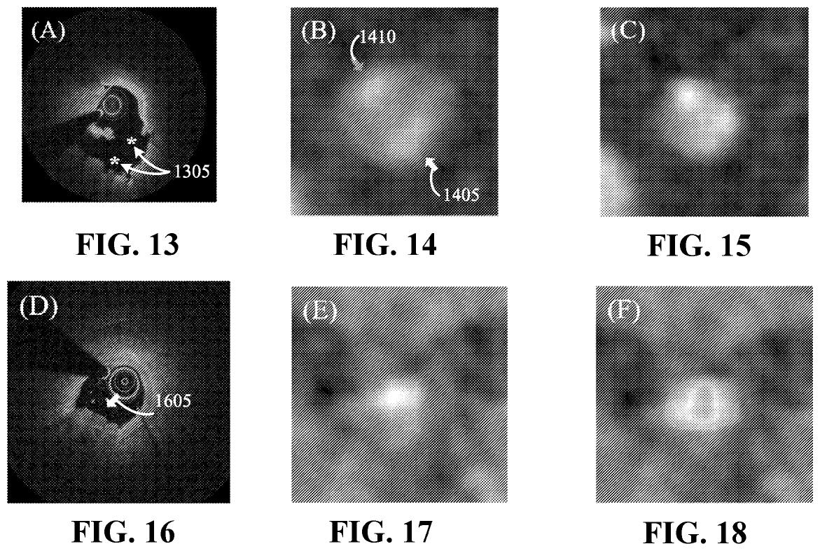

The attention of the slice-level and sequence-level transformer, which reflect the attention of the model within the slice and between the slices, respectively, may be visualized. As provided in the representative cases in FIGS. 13-18, the DL model paid attention accurately to the lesion location compared to the ground truth annotation at the patient-level. Within a single frame, the suspected culprit lesion was well localized by the model attention, suggesting that the model can identify the clinically important features within the given frame.

FIG. 13 is an OCT image of plaque rupture. Plaque rupture may be characterized by the presence of fibrous cap discontinuity with a cavity formation within the plaque. Cavity formation within the plaque is represented in FIG. 13 by asterisks labeled with reference numerals 1305. FIGS. 14-15 are CTA images of the corresponding site. The CTA image of FIG. 14 shows a contrast effect at two locations: the site of the cavity (represented in FIG. 14 by arrow labeled with reference numeral 1405) observed on the OCT image and the vessel lumen (represented in FIG. 14 by arrow labeled with reference numeral 1410). The CTA image of FIG. 15 shows that the DL model focuses on the two contrast effects illustrated in FIG. 14. FIG. 16 is an OCT image of plaque erosion. Definite plaque erosion may be characterized by the presence of attached thrombus (represented in FIG. 16 by the arrow labeled with reference numeral 1605) overlying an intact and visualized plaque. FIGS. 17-18 are CTA images of the corresponding site. The CTA image of FIG. 17 shows a small lumen surrounded by plaque without a cavity. The CTA image of FIG. 18 shows that the DL model was concentrated at the site of stenosis without evidence of a cavity.

3.5—Reader Study Comparing the Model Performance with Experienced Cardiologists.

In the first round of the reader study, the performances of the MD-CTA model were compared with the experienced cardiologists, as shown in Table 10 (below).

TABLE 10
Results of the reader study to validate the clinical utility of the deep learning model

| | Sensitivity | Specificity | | | FPR | FNR |
|---|---|---|---|---|---|---|
| | (%) | (%) | PPV | NPV | (%) | (%) |
| | (95% CI) | (95% CI) | (95% CI) | (95% CI) | (95% CI) | (95% CI) |
| Novel DL | 87.1 | 85.3 | 71.1 | 94.1 | 14.7 | 12.9 |
| model | (70.2-96.4) | (75.3-92.4) | (58.3-81.2) | (86.5-97.6) | (7.6-24.7) | (3.6-29.8) |

Before DL model assistance

| | | | | | | |
|---|---|---|---|---|---|---|
| Reader 1 | 16.1 | 77.3 | 22.7 | 69.1 | 22.7 | 83.9 |
| | (5.5-33.7) | (66.2-86.2) | (10.6-42.1) | (64.7-73.1) | (13.8-33.8) | (66.3-94.5) |
| Reader 2 | 12.9 | 88.0 | 30.8 | 71.0 | 12.0 | 87.1 |
| | (3.6-29.8) | (78.4-94.4) | (12.9-57.2) | (67.6-74.1) | (5.6-21.6) | (70.2-96.4) |
| Reader 3 | 16.1 | 76.0 | 21.7 | 68.7 | 24.0 | 83.9 |
| | (5.5-33.7) | (64.8-85.1) | (10.2-40.5) | (64.2-72.8) | (14.9-35.2) | (66.3-94.5) |

After DL model assistance

| | | | | | | |
|---|---|---|---|---|---|---|
| Reader 1 | 83.9 | 89.3 | 76.5 | 93.1 | 10.7 | 16.1 |
| | (66.3-94.6) | (80.1-95.3) | (62.4-86.4) | (85.7-96.8) | (4.7-9.9) | (5.4-33.7) |
| Reader 2 | 77.4 | 85.3 | 68.6 | 90.1 | 14.7 | 22.6 |
| | (58.9-90.4) | (75.3-92.4) | (55.0-79.6) | (82.6-94.6) | (7.6-24.7) | (9.6-41.1) |
| Reader 3 | 77.4 | 85.3 | 68.6 | 90.1 | 14.7 | 22.6 |
| | (58.9-90.4) | (75.3-92.4) | (55.0-79.6) | (82.6-94.6) | (7.6-24.7) | (9.6-41.1) |

As shown in Table 10, the model outperformed the expert readers in all diagnostic performance metrics, and the superiority of the model was most prominent for the sensitivity (87.1% in DL model vs. 16.1% in reader 1, 12.9% in reader 2, and 16.1% in reader 3). The second round of the reader study was performed to evaluate whether the model can be used as an assisting tool to improve the diagnostic performance of the human reader. When given the prediction results of the model prediction results for the probability and location of the plaque erosion, the diagnostic performances of the human readers markedly improved, especially for the sensitivity, increasing from 16.1% to 83.9% in reader 1, from 12.9% to 77.4% in reader 2, and from 16.1% to 77.4% in reader 3.

### 4—Discussion

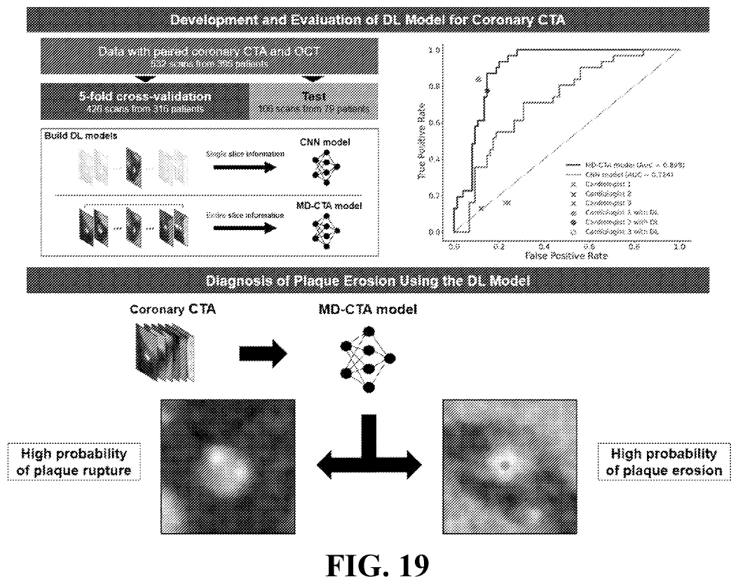

As provided herein, and generally illustrated in FIG. 19, the technology disclosed herein provides methods and systems for providing automated diagnosis of plaque erosion with a non-invasive coronary CTA using a DL model (also referred to herein as the “MD-CTA model”). The MD-CTA model disclosed herein may leverage composite transformer attentions to incorporate the information and relationships between the coronary CTA slices. In some configurations, the technology disclosed herein may implement modality-specific self-supervised pre-training to further enhance the performance of the MD-CTA model. The five-fold cross-validation and the test set validation results discussed herein shown that the DL model can diagnose plaque erosion solely from the CTA images, attaining a clinically useful level of diagnostic performance. The model disclosed herein outperformed experienced cardiologists, and, when used as an assisting tool, the diagnostic performances of cardiologists were markedly improved.

Plaque erosion, which is responsible for up to 50% of patients with non-ST-segment elevation (NSTE)-ACS, is characterized by an intact fibrous cap, preserved vascular structure, and platelet-rich thrombus. Thrombus in plaque erosion is attributed to apoptosis or denudation of superficial endothelial cells as opposed to fibrous cap disruption and creation of a cavity inside a plaque in plaque rupture. ACS patients with plaque erosion may have fewer cardiovascular risk factors, less atherosclerotic burden and lower frequency of complex lesions, less multivessel coronary artery disease, and higher prevalence of close proximity to a bifurcation than those with plaque rupture. In addition, on OCT images, patients with plaque erosion may have smaller reference vessel diameter, lower prevalence of calcification and thrombus in culprit lesions, and lower prevalence of macrophage accumulation, microvessels, and spotty calcium in non-culprit lesions than those with plaque rupture. This may suggest that plaque erosion is associated with lower levels of pan-vascular vulnerability and exhibits rather subtle structural changes at the microscopic level. When the aforementioned microscopic structural changes can be identified by using DL, plaque erosion can be diagnosed by these specific findings, rather be diagnosed by excluding plaque rupture, as it currently stands. Since patients with NSTE-ACS can usually be stabilized with medical therapy and preliminary data suggest that conservative management might be an option for ACS patients caused by plaque erosion, by make a diagnosis of plaque erosion by using CTA using the technology disclosed herein, this subset of patients might be able to be managed without invasive procedures.

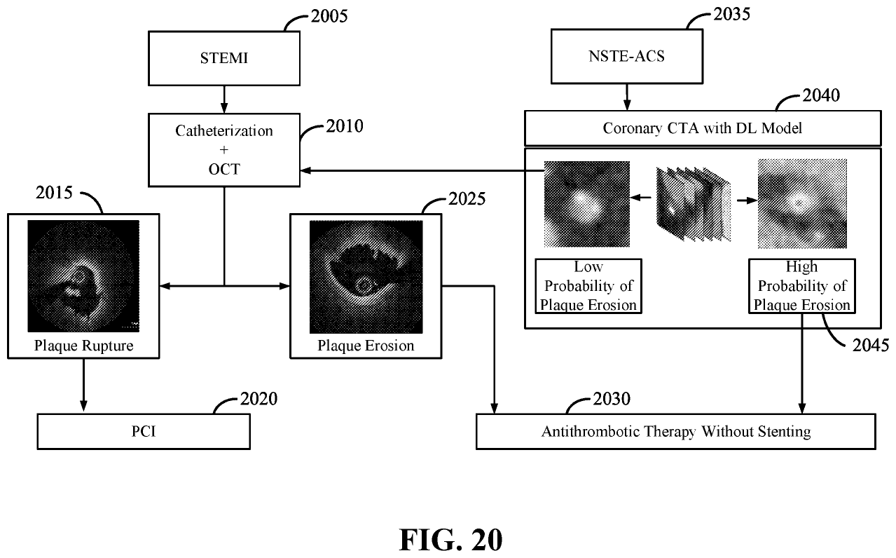

FIG. 20 illustrates an example approach for evaluation and management of patients with ACS. As illustrated in FIG. 20, patients with ST-segment elevation myocardial infarction (STEMI) (represented in FIG. 20 by reference numeral 2005) would undergo emergency catheterization (represented in FIG. 20 by reference numeral 2010). If plaque rupture is confirmed (represented in FIG. 20 by reference numeral 2015), the culprit lesion would be treated with stenting or another type of percutaneous coronary intervention (PCI) (represented in FIG. 20 by reference numeral 2020). If OCT demonstrated plaque erosion with preserved lumen (represented in FIG. 20 by reference numeral 2025), antithrombotic therapy without stenting could be considered (represented in FIG. 20 by reference numeral 2030). Patients with NSTE-ACS (represented in FIG. 20 by reference numeral 2035) may undergo noninvasive coronary CTA with DL model after stabilization (represented in FIG. 20 by reference numeral 2040). If there is high probability of plaque erosion and preserved lumen (represented in FIG. 20 by reference numeral 2045), antithrombotic therapy without stenting could be considered (represented in FIG. 20 by reference numeral 2030).

The challenge with CTA is its capability to detect the subtle structural changes that occur in plaque erosion due to the lower resolution of CTA. The technology disclosed herein successfully surmounted this conundrum. First, the technology disclosed herein may use a design with composite transformer attention along with the modality-specific pre-training method to improve the overall performance of the model. Second, the technology disclosed herein may utilize the unique database containing paired coronary CTA and OCT images obtained from each patient, which enables building the model for CTA supervised with an OCT-based label as the gold standard, attaining a clinically useful level of diagnostic performance with only CTA scans. Compared to the conventional CNN model, the MD-CTA model disclosed herein significantly improved the AUC from 0.724 to 0.899.

In recent years, there have been more than 800,000 patients with myocardial infarction in the United States per year and NSTEMI has recently become the most frequent type of MI (NSTEMI increased from 52.8% in 2002 to 68.6% in 2011). In patients with NSTEMI, plaque erosion is the underlying pathology in up to 75% of cases. Thus, the potential number of patients who might benefit from the technology disclosed herein is enormous.

### 4.1—Study Considerations.

The study disclosed herein had several considerations. First, the test set validation was performed for the randomly split subset from the single data source. To alleviate concerns for the generalizability, vendor-specific pre- or post-processing was not utilized, and the raw Hounsfield Unit values were used as the input of the model after simple normalization between 0-1. Second, instead of histological ground truth, the concurrent OCT images that have higher resolution were leveraged as the gold standard. This approach was adopted since histological diagnosis in living patients is generally unavailable. This approach has been widely adopted in developing models for medical image analysis when histological validation is not feasible. Third, less common ACS pathologies, such as a calcified plaque, spontaneous coronary dissection, and intraplaque hemorrhage were excluded. Fourth, the diagnostic accuracy of the model may be affected by a quality of an image. For instance, severely calcified plaque or severe luminal narrowing may lower the accuracy of the diagnosis. Of note, plaque erosion, compared to plaque rupture, in general has a larger lumen. Fifth, although the unique and well-curated dataset consisting of paired coronary CTA and OCT may be used, the size of the dataset may still be small. Although large scale studies with clinical outcomes would be helpful, combined pre-procedure CTA and intracoronary imaging in the same patients with ACS would be practically challenging.

### 5—Conclusions

The MD-CTA model, specifically designed for coronary CTA and trained with the paired coronary CTA and OCT database, can accurately diagnose plaque erosion using non-invasive coronary CTA images and significantly outperformed experienced cardiologists, and, as such, the technology disclosed herein may lead to a major shift in the management of millions of patients with ACS each year.

### 6—Systems and Methods

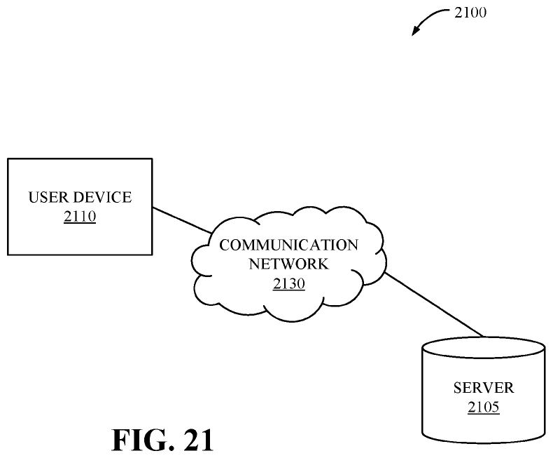

FIG. 21 illustrates a system 2100 for diagnosing plaque erosion using coronary CTA with deep learning model(s) according to some configurations. In the illustrated example, the system 2100 includes a server 2105 and a user device 2110. In some configurations, the system 2100 includes fewer, additional, or different components than illustrated in FIG. 21. As one example, the system 2100 may include multiple servers 2105, multiple user devices 2110, or a combination thereof. As another example, the system 2100 may include a medical imaging system or modality (e.g., a CTA imaging system), a medical database, etc. As yet another example, one or more components of the system 2100 may be combined into a single device, such as, e.g., the server 2105 and the user device 2110. As one example, the functionality (or a portion thereof) described herein as being performed by the server 2105 may be performed by the user device 2110.

The server 2105 and the user device 2110 communicate over one or more wired or wireless communication networks 2130. Portions of the communication networks 2130 may be implemented using a wide area network, such as the Internet, a local area network, such as a Bluetooth™ network or Wi-Fi, and combinations or derivatives thereof. Alternatively, or in addition, in some configurations, components of the system 2100 communicate directly as compared to through the communication network 2130. Also, in some configurations, the components of the system 2100 communicate through one or more intermediary devices not illustrated in FIG. 21.

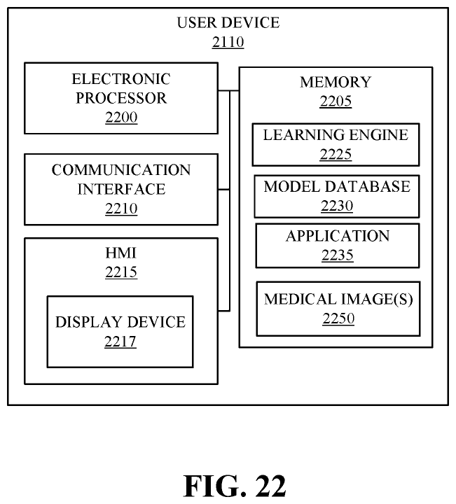

The user device 2110 includes a computing device, such as a desktop computer, a laptop computer, a tablet computer, a terminal, a smart telephone, a smart television, a smart wearable, or another suitable computing device that interfaces with a user. FIG. 22 schematically illustrates an example user device 2110 according to some configurations. As illustrated in FIG. 22, the user device 2110 may include an electronic processor 2200, a memory 2205, a communication interface 2210, and a human-machine interface (“HMI”) 2215. The electronic processor 2200, the memory 2205, the communication interface 2210, and the HMI 2215 may communicate wirelessly, over one or more communication lines or buses, or a combination thereof. The user device 2110 may include additional, different, or fewer components than those illustrated in FIG. 22 in various configurations. The user device 2110 may perform additional functionality other than the functionality described herein. Also, the functionality (or a portion thereof) described herein as being performed by the user device 2110 may be performed by another component (e.g., the server 2105, another computing device, another component of the system 2100, etc.), distributed among multiple computing devices (e.g., as part of a cloud service or cloud-computing environment), combined with another component (e.g., the server 2105, another computing device, another component of the system 2100, etc.), or a combination thereof.

The communication interface 2210 may include a transceiver that communicates with the server 2105, another user device or component of the system 2100, or a combination thereof over the communication network 2130 and, optionally, one or more other communication networks or connections. The electronic processor 2200 includes a microprocessor, an application-specific integrated circuit (“ASIC”), or another suitable electronic device for processing data, and the memory 2205 includes a non-transitory, computer-readable storage medium. The electronic processor 2200 is configured to retrieve instructions and data from the memory 1505 and execute the instructions.

As illustrated in FIG. 22, the user device 2110 may also include the HMI 2215 for interacting with a user. The HMI 2215 may include one or more input devices, one or more output devices, or a combination thereof. Accordingly, in some configurations, the HMI 2215 allows a user to interact with (e.g., provide input to and receive output from) the user device 2110. For example, the HMI 2215 may include a keyboard, a cursor-control device (e.g., a mouse), a touch screen, a scroll ball, a mechanical button, a display device (e.g., a liquid crystal display (“LCD”)), a printer, a speaker, a microphone, or a combination thereof.

In the illustrated example of FIG. 22, the HMI 2215 includes a display device 2217. The display device 2217 may be included in the same housing as the user device 2110 or may communicate with the user device 2110 over one or more wired or wireless connections. As one example, the display device 2217 may be a touchscreen included in, e.g., a laptop computer, a tablet computer, a smart telephone, or the like. As another example, the display device 2217 may be, e.g., a monitor, a television, a projector, or the like coupled to a terminal, desktop computer, or the like via one or more cables.

As illustrated in FIG. 22, the memory 2205 may store a learning engine 2225 and a model database 2230. In some configurations, the learning engine 2225 develops one or more models using one or more machine learning functions. Machine learning functions are generally functions that allow a computer application to learn without being explicitly programmed. In particular, the learning engine 2225 is configured to develop an algorithm or model based on training data. As one example, to perform supervised learning, the training data includes example inputs and corresponding desired (for example, actual) outputs, and the learning engine 2225 progressively develops a model that maps inputs to the outputs included in the training data. Machine learning performed by the learning engine 2225 may be performed using various types of methods and mechanisms including but not limited to decision tree learning, association rule learning, artificial neural networks, inductive logic programming, support vector machines, clustering, Bayesian networks, reinforcement learning, representation learning, similarity and metric learning, sparse dictionary learning, and genetic algorithms. These approaches allow the learning engine 1525 to ingest, parse, and understand data and progressively refine models.

As described in greater detail herein, in some configurations, the technology disclosed herein may utilize or implement one or more models or algorithms as part of diagnosing plaque erosion using coronary CTA (referred to herein as the DL model or the MD-CTA model). Accordingly, in some configurations, the learning engine 2225 may be used to train one or more of those models or algorithms. For instance, the learning engine 2225 may utilize the methods disclosed herein to develop, maintain, and train the DL model (or the MD-CTA model).

The model(s) generated by the learning engine 2225 can be stored in the model database 2230. As illustrated in FIG. 22, the model database 2230 is included in the memory 2205 of the user device 2110. It should be understood, however, that, in some configurations, the model database 2230 is included in a separate device accessible by the user device 2110 (including a remote database, the server 2105, or the like).

As illustrated in FIG. 22, the memory 2205 may include one or more medical images 2250. The medical image(s) 2250 may include medical images or scans as described herein. For instance, in some configurations, the medical image(s) 2250 may include coronary CTA images (or scans), as described in greater detail herein. While the medical image(s) 2250 are illustrated in FIG. 22 as being included in the memory 2205 of the user device 2110, it should be understood, however, that, in some configurations, the medical image(s) 2250 (or a portion thereof) may be included (or otherwise stored) in a separate device accessible by the user device 2110 (including a remote database, the server 2105, or the like).

As also illustrated in FIG. 22, the memory 2205 may include an application 2235. The application 2235 is a software application executable by the electronic processor 2200 in the example illustrated and as specifically discussed below, although a similarly purposed module can be implemented in other ways in other examples. In some configurations, the application 2235 may be a dedicated software application locally stored in the memory 2205 of the user device 2110. Alternatively, or in addition, the application 2235 may be remotely hosted and accessible from the server 2105 (e.g., separate from the user device 2110 of FIG. 21), such as where the application 2235 is (or enables) a web-based service or functionality. As described in greater detail herein, the application 2235 (when executed by the electronic processor 2200) may enable or facilitate diagnosing plaque erosion using coronary CTA, and, in some instances, diagnose plaque erosion using one or more models stored in the model database 2230 (e.g., the MD-CTA model). As one example, the application 2235 (when executed by the electronic processor 2200) may access one or more models or algorithms stored in the model database 2230 and diagnose plaque erosion using the one or more models or algorithms such that the application 2235 provides a deep learning based plaque erosion diagnosis using coronary CTA.

The memory 2205 may include additional, different, or fewer components in different configurations. Alternatively, or in addition, in some configurations, one or more components of the memory 2205 may be combined into a single component, distributed among multiple components, or the like. Alternatively, or in addition, in some configurations, one or more components of the memory 2205 may be stored remotely from the user device 2110, or, in a remote database, a remote server, another user device, an external storage device, or the like.

Returning to FIG. 21, the system 2100 may also include at least one server 2105. The server 2105 may include a computing device, such as a server, a database, or the like. The server 2105 may host or otherwise provide a service or platform associated with the application 2235. In some examples, the server 2105 may host a service for using deep learning to diagnose plaque erosion using coronary CTA. Accordingly, in some configurations, the server 2105 is associated with the application 2235.

Although not illustrated in FIG. 21, the server 2105 may include similar components as the user device 2110, such as electronic processor (for example, a microprocessor, an ASIC, or another suitable electronic device), a memory (for example, a non-transitory, computer-readable storage medium), a communication interface, such as a transceiver, for communicating over the communication network 2130 and, optionally, one or more additional communication networks or connections, and one or more human machine interfaces. In some configurations, the functionality (or a portion thereof) as described as being performed by the server 2105 may be locally performed by the user device 2110. As one example, in some configurations, the user device 2110 may host or provide at least one application platform. In such configurations, the server 2105 may be eliminated from the system 2100. Alternatively, or in addition, in some configurations, the server 2105 may perform additional or different functionality than described herein. As one example, in some configurations, the functionality (or a portion thereof) as being performed by the user device 2110 may be performed by the server 2105. In such configurations, the server 2105 may store at least one of, e.g., the application 2235, the learning engine 2225, the model database 2230, or the like.

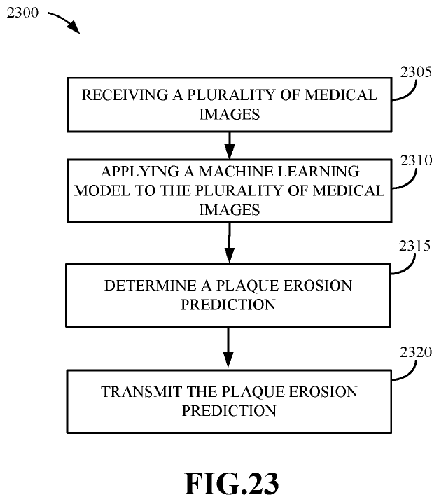

FIG. 23 is a flowchart illustrating a method 2300 for diagnosing plaque erosion using coronary CTA according to some configurations. The method 2300 is described as being performed by the user device 2110 and, in particular, the application 2235 as executed by the electronic processor 2200. However, as noted above, the functionality (or a portion thereof) described with respect to the method 2300 may be performed by other devices, such as the server 2105, or distributed among a plurality of devices (e.g., distributed among the user device 2110 and the server 2105), such as a plurality of servers included in a cloud service.

As illustrated in FIG. 23, the method 2300 may include receiving a plurality of medical images (at block 2305). In some configurations, the electronic processor 200 may receive (or otherwise access) the medical image(s) 2250 from the memory 2205. Alternatively, or in addition, in some configurations, the electronic processor 200 may receive (or otherwise access) the medical image(s) 2250 from a separate, remote device, such as, e.g., the server 2105, a remote database, etc. As described in greater detail herein, in some configurations, the medical image(s) 2250 may include coronary CTA images (or scans). In some examples, the medical image(s) 2250 may be for a patient having ACS.

The electronic processor 2200 may apply a machine learning model to the medical images (at block 2310) and determine a plaque erosion prediction based on application of the machine learning model to the plurality of CTA images (at block 2315). The machine learning model may include, e.g., the DL model, the MD-CTA model, another model described herein. In some configurations, the electronic processor 2200 may access (or otherwise retrieve) the machine learning model from the model database 2230. Alternatively, or in addition, in some configurations, the electronic processor 2200 may access (or otherwise retrieve) the machine learning model from another location or device (e.g., the server 2105, etc.). In some configurations, the machine learning model may be a modality-specific deep learning model trained using machine learning to diagnose plaque erosion using coronary CTA, as described in greater detail herein. In some examples, the machine learning model may include a composite transformer attention component and a momentum distillation component as described in greater detail herein. Alternatively, or in addition, in some examples, the machine learning model may include a spatial transformer, a sequential transformer, or a combination thereof, as also described in greater detail herein.

In some configurations, prior to applying the machine learning model to the medical images (e.g., at block 2310), the electronic processor 2200 may train the machine learning model using one or more methods or processes described herein. For instance, in some configurations, the electronic processor 2200 may execute (or otherwise invoke) the learning engine 2225 to train the machine learning model using, e.g., training data, as described in greater detail herein. In some configurations, the training data may include, e.g., a plurality of training CTA images, where a portion of the training CTA images are annotated. Alternatively, or in addition, in some configurations, as described in greater detail herein, the electronic processor 2220 may perform momentum distillation-enhanced self-supervised pre-training with respect to the machine learning model.

After accessing the machine learning model, the electronic processor 2200 may apply the machine learning model by, e.g., providing the medical images 2250 to the machine learning model (as an input). Responsive to receiving the medical images 2250 the machine learning model may determine a plaque erosion prediction (as an output).

In some configurations, the electronic processor 2200 may apply a spatial transformer of the machine learning model to each slice (e.g., CTA slice) included in each medical image 2250. Based on the application of the spatial transformer, the electronic processor 2200 (e.g., the spatial transformer of the machine learning model) may generate a corresponding feature vector for each of the slices. The corresponding feature vectors may be provided to a sequential transformer of the machine learning model. Upon receipt of the corresponding feature vectors, the electronic processor 2200 (e.g., the spatial transformer of the machine learning model) may generate and output a transformed feature. As described in greater detail herein, in some configurations, the electronic processor 2200 may determine the plaque erosion prediction based on the transformed feature.

Accordingly, in some configurations, the electronic processor 2200 may determine, with the machine learning model, a slice-level prediction based on the medical images 2250 (e.g., using the spatial transformer of the machine learning model). The electronic processor 220 may determine, with the machine learning model, a patient-level prediction based on the medical images 2250 (e.g., using the sequential transformer of the machine learning model). In some instances, the plaque erosion prediction may be based on the slice-level prediction, the patient-level prediction, or a combination thereof.

In some configurations, the electronic processor 2200 may transmit the plaque erosion prediction for display via, e.g., the display device 2217 (at block 2320). In some configurations, the electronic processor 2200 may determine a recommended treatment (or course of treatment) based on the plaque erosion prediction, as described in greater detail herein (e.g., with respect to at least FIG. 20). As one example, when the plaque erosion prediction indicates a first probability of plaque erosion (e.g., a high probability of plaque erosion), the recommended course of treatment may include an antithrombotic therapy without stenting. As another example, when the plaque erosion prediction indicates a second probability of plaque erosion (e.g., a low probability of plaque erosion), the recommended course of treatment may include a percutaneous coronary intervention. In some configurations, the electronic processor 2200 may transmit the recommended treatment for display via the display device 2217.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), Flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the technology disclosed herein.

1. A system (2100) for implementing deep learning to noninvasively diagnose plaque erosion using coronary computed tomography angiography (CTA), the system (2100) comprising:
one or more electronic processors (2200) configured to:
receive a plurality of CTA images (2250) for a patient having acute coronary syndromes (ACS);
apply a machine learning model to the plurality of CTA images (2250), wherein the machine learning model is a modality-specific deep learning model trained using machine learning to diagnose plaque erosion using coronary CTA;
determine a plaque erosion prediction based on application of the machine learning model to the plurality of CTA images (2250); and
transmit the plaque erosion prediction for display via a display device (2217).

2. The system (2100) of claim 1, wherein the one or more electronic processors (2200) are configured to:
determine, based on the plaque erosion prediction, a recommended course of treatment for the patient; and
transmit the recommended course of treatment for display via the display device (2217).

3. The system (2100) of claim 2, wherein the recommended course of treatment includes an antithrombotic therapy without stenting when the plaque erosion prediction indicates a first probability of plaque erosion; and
wherein the recommended course of treatment includes a percutaneous coronary intervention when the plaque erosion prediction indicates a second probability of plaque erosion, wherein the second probability of plaque erosion is lower than the first probability of plaque erosion.

4. The system (2100) of claim 1, wherein the plaque erosion prediction includes at least one of a probability of plaque erosion or a probability of plaque rupture.

5. The system (2100) of claim 1, wherein the machine learning model includes a composite transformer attention component and a momentum distillation component.

6. The system (2100) of claim 1, wherein the machine learning model includes a spatial transformer and a sequential transformer.

7. The system (2100) of claim 1, wherein the one or more electronic processors (2200) are configured to determine the plaque erosion prediction by:
applying a spatial transformer of the machine learning model to each of a plurality of CTA slices included in each of the plurality of CTA images (2250) to generate a corresponding feature vector for each of the plurality of CTA slices; and
applying a sequential transformer of the machine learning model to each corresponding feature vector of each of the plurality of CTA slices to output a transformed feature,
wherein the machine learning model is configured to determine the plaque erosion prediction based on the transformed feature.

8. The system (2100) of claim 1, wherein the one or more electronic processors (2200) are configured to:
determine, with the machine learning model, a slice-level prediction based on the plurality of CTA images (2250); and
determine, with the machine learning model, a patient-level prediction based on the plurality of CTA images (2250),
wherein the plaque erosion prediction is based on the slice-level prediction and the patient-level prediction.

9. The system (2100) of claim 1, wherein the one or more electronic processors (2200) are configured to:
access training data including a plurality of training CTA images, wherein at least a portion of the plurality of training CTA images are annotated; and
train, with the training data, the machine learning model using momentum distillation-enhanced self-supervised pre-training.

10. A method (2300) for implementing deep learning to noninvasively diagnose plaque erosion using coronary computed tomography angiography (CTA), the method comprising:
receiving, with one or more electronic processors (2200), a plurality of medical images (2250);
applying, with the one or more electronic processors (2200), a machine learning model to the plurality of medical images (2250), wherein the machine learning model is a modality-specific deep learning model including a spatial transformer and a sequential transformer;
determining, with the one or more electronic processors (2200), a plaque erosion prediction based on application of the machine learning model to the plurality of medical images (2250); and
transmitting, with the one or more electronic processors (2200), the plaque erosion prediction for display via a display device (2217).

11. The method (2300) of claim 10, further comprising:
performing, with the one or more electronic processors (2200), momentum distillation-enhanced self-supervised pre-training of the machine learning model prior to receiving the plurality of medical images (2250).

12. The method (2300) of claim 10, wherein receiving the plurality of medical images (2250) includes receiving a plurality of coronary CTA images for a patient that has acute coronary syndromes (ACS).

13. The method (2300) of claim 10, wherein applying the machine learning model to the plurality of medical images (2250) includes:
applying, with the one or more electronic processors (2200), a spatial transformer of the machine learning model to each of a plurality of slices included in each of the plurality of medical images (2250) to generate a corresponding feature vector for each of the plurality of slices; and
applying, with the one or more electronic processors (2200), a sequential transformer of the machine learning model to each corresponding feature vector of each of the plurality of slices to output a transformed feature,
wherein determining the plaque erosion prediction includes determining the plaque erosion prediction based on the transformed feature.

14. A non-transitory, computer-readable medium (2205) storing instructions that, when executed by one or more electronic processors (2200), perform a set of functions, the set of functions comprising:
receiving a plurality of CTA images (2250);
applying, to the plurality of CTA images (2250), a modality-specific deep learning model including a spatial transformer and a sequential transformer, wherein applying the modality-specific deep learning model includes:
generating, via the spatial transformer, a corresponding feature vector for each of a plurality of CTA slices included in each of the plurality of CTA images (2250); and
outputting, via the sequential transformer, a transformed feature based on each corresponding feature vector of each of the plurality of CTA slices;
determining a plaque erosion prediction based on application of the modality-specific deep learning model to the plurality of CTA images (2250); and
transmitting the plaque erosion prediction for display via a display device (2217).

15. The non-transitory, computer-readable medium (2205) of claim 14, the set of functions further comprising:
determining a recommended treatment based on the plaque erosion prediction; and
outputting the recommended treatment,
wherein, when the plaque erosion prediction indicates a first probability of plaque erosion, the recommended treatment includes an antithrombotic therapy without stenting, and
wherein, when the plaque erosion prediction indicates a second probability of plaque erosion, the recommended treatment includes a percutaneous coronary intervention, wherein the second probability of plaque erosion is lower than the first probability of plaque erosion.
